(19) United States
(12) Patent Application Publication
(10) Pub. No.: US 2012/0231143 A1
(43) Pub. Date: Sep. 13, 2012

(54) VIRUS-INACTIVATING AGENT

(75) Inventors: Hiroo Kawakami, Hyogo (JP); Hirohide Kotani, Osaka (JP); Noriko Kotani, Osaka (JP); Yasuharu Yamashita, Osaka (JP)

(73) Assignee: Kawakami Co. Ltd, Osaka (JP)

(21) Appl. No.: 13/508,603

(22) PCT Filed: Nov. 9, 2010
(86) PCT No.: PCT/JP2010/069919

§ 371 Date: May 8, 2012

(30) Foreign Application Priority Data
Nov. 10, 2009 (JP) 2009 257384

(51) Int. Cl. C11B 5/00; A61P 31/12; A61K 9/00; A61K 33/08

(52) U.S. Cl. 426/541; 424/693; 424/400

(57) ABSTRACT

The virus-inactivating effect of calcinated calcium and the like is elucidated, and a novel virus-inactivating agent particularly having a norovirus-inactivating effect is provided. The virus-inactivating agent comprises an aqueous solution or dispersion which is prepared by adding at least one component selected from calcium hydroxide, calcium oxide and calcinated calcium to water. In addition to the above-mentioned components, the virus-inactivating agent may additionally comprise ethanol and a salt of an organic acid.

### BACKGROUND OF THE INVENTION

1. Field of the Invention

The present invention relates to a virus-inactivating agent and particularly relates to a virus-inactivating agent having inactivating effects against norovirus and influenza A virus.

2. Description of the Related Art

Since previously, compositions having an alcohol as a main agent have been widely used as microbe eradicating agents, and further as microbe eradicating agents having an inactivating effect against norovirus, a composition in which a grapefruit seed extract and phytic acid are blended with brewer's alcohol (Patent Document 1), a composition in which an alkaline substance and a surfactant are blended with ethanol (Patent Document 2) , and a composition in which an organic acid salt and a eucalyptus extract are blended with ethanol (Patent Document 3), etc., have been disclosed.

Meanwhile recently, it has been disclosed that calcinated shell calcium, calcium oxide, and calcium hydroxide as a hydrate of calcium oxide, have antimicrobial properties (Patent Document 4), and a microbe suppressing agent for foods in which an organic acid salt is blended with calcinated shell calcium (Patent Document 5), a microbe eradicating agent for foods in which a polyvalent alcohol fatty acid ester and ethanol are blended with calcinated calcium (Patent Document 6), etc. have been disclosed. However, an inactivating effect of calcinated shell calcium, etc. against norovirus was unknown.

Patent Document 1: Japanese Published Unexamined Patent Application: No. 2007-320924

Patent Document 2: Japanese Published Unexamined Patent Application: No. 2008-189645

Patent Document 3: Japanese Published Unexamined Patent Application: No. 2009-179577

Patent Document 4: Japanese Published Unexamined Patent Application: No. H11-222796

Patent Document 5: Japanese Published Unexamined Patent Application: No. H11-290044

Patent Document 6: Japanese Published Unexamined Patent Application: No. 2002-272434

### SUMMARY OF THE INVENTION

The present invention has been made in view of the above circumstances and objects thereof are to elucidate virus-inactivating effects of calcinated calcium, etc., and provide a novel virus-inactivating agent having, in particular, a norovirus-inactivating effect.

As a result of conducting diligent research, the present inventors found that an aqueous solution or aqueous dispersion in which calcium hydroxide is blended not only has a norovirus-inactivating effect but also has an influenza A virus-inactivating effect, and have thereby come to complete the present invention.

That is, in gist, the present invention provides the following.
[1] A norovirus-inactivating agent that is an aqueous solution or aqueous dispersion prepared by blending in calcium hydroxide.
[2] An influenza A virus-inactivating agent that is an aqueous solution or aqueous dispersion prepared by blending in calcium hydroxide.
[3] The virus-inactivating agent according to [1] or [2] prepared by blending in calcium oxide and/or calcinated calcium in place of calcium hydroxide or in addition to calcium hydroxide.
[4] The virus-inactivating agent according to any one of [1] to [3] prepared by further blending in ethanol and sodium lactate.
[5] The virus-inactivating agent according to [4] where a total blending proportion of the blended components among calcium hydroxide, calcium oxide, and calcinated calcium is 0.01 to 30 weight %, a blending proportion of ethanol is 5 to 30 weight %, and a blending proportion of sodium lactate is 1 to 30 weight %.
[6] The virus-inactivating agent according to any one of [1] to [5] where an average particle diameter of calcium hydroxide, calcium oxide, and calcinated calcium is 0.1 to 10 μm.
[7] A food obtained by virus inactivation treatment by the virus-inactivating agent according to any one of [1] to [6] .
[8] The food according to [7] that is a fresh fish or shellfish.

By the present invention, a virus-inactivating agent having a norovirus-inactivating effect and an influenza A virus-inactivating effect is provided.

### BRIEF DESCRIPTION OF THE DRAWINGS

FIG. 1 is an electrophoretogram of PCR products;

FIG. 2 is an electrophoretogram of PCR products;

FIG. 3 is a diagram of variations with time of viable bacterial counts in raw oysters contaminated with Staphylococcus aureus when a microbe eradication treatment was applied to the raw oysters FIG. 4 is a diagram of variations with time of viable bacterial counts in raw oysters contaminated with Salmonella enteritidis when a microbe eradication treatment was applied to the raw oysters;

FIG. 5 is a diagram of variations with time of viable bacterial counts in raw oysters contaminated with enterohemorrhagic Escherichia coli O-157 when a microbe eradication treatment was applied to the raw oysters;

FIG. 6 is a diagram of variations with time of viable bacterial counts in raw oysters contaminated with Vibrio parahaemolyticus when a microbe eradication treatment was applied to the raw oysters;

FIG. 7 is a diagram of variations with time of viable bacterial counts in raw oysters contaminated with Escherichia coli when a microbe eradication treatment was applied to the raw oysters; and

FIG. 8 is a diagram of variations with time of viable bacterial counts in raw oysters immersed in artificial seawater when a microbe eradication treatment was applied to the raw oysters.

### DETAILED DESCRIPTION OF THE PREFERRED EMBODIMENTS

The virus inactivating agent according to the present invention is an aqueous solution or aqueous dispersion obtained by blending calcium hydroxide with water and has a norovirus-inactivating effect, an influenza A virus-inactivating effect, and a feline calicivirus-inactivating effect and further has a microbe eradicating effect against food poisoning microbes in general.

As blended components of the present invention, one or more of a component that reacts with water to produce calcium hydroxide, for example, calcium oxide, calcinated calcium, etc., may be used in place of calcium hydroxide or in addition to calcium hydroxide. Hereinafter in the present specification, calcium hydroxide and components, such as calcium oxide, calcinated calcium, etc., that react with water to produce calcium hydroxide, shall be referred to collectively as “virus-inactivating components.”

Among the virus-inactivating components, calcinated calcium is composed mainly of calcium oxide and is obtained by subjecting animal-derived calcium which are composed of calcium carbonate before calcination, such as oyster shells, scallop shells, surf clam shells, eggshells, or coral shells, to calcination or ohmic heating at a temperature of no less than 600° C. and preferably 900 to 1200° C. for approximately 15 to 60 minutes. Preferably, a saturated aqueous solution of the calcinated calcium obtained has a pH in a range of 11 to 13. As the calcinated calcium, that which complies with food additive standards is normally used.

An average particle diameter of the virus-inactivating components is not restricted in particular and is normally 0.1 to 10 μm. Also, a blending proportion of the virus-inactivating components in the virus-inactivating agent is not restricted in particular and is normally 0.01 to 30 weight %.

With the present invention, as other blended components besides the virus-inactivating components, for example, ethanol or an organic acid salt may be blended to increase the virus-inactivating effect or the microbe eradicating effect against food poisoning microbes. As the ethanol, that which complies with food additive standards is normally preferable. A blending proportion of the ethanol in the virus-inactivating agent is not restricted in particular, and the ethanol is normally blended in a range of 5 to 30 weight %.

Examples of the organic acid salt include citrates, lactates, malates, tartrates, oxalates, acetates, formates, succinates, fumarates, gluconates, etc., and these may be used solely or in suitable combination of two or more thereof. Examples of the salt include an alkali metal salt, etc. Among the above, lactates are preferable and sodium lactates are more preferable. A blending proportion of the organic acid salt in the virus-inactivating agent is not restricted in particular, and the organic acid salt is normally blended in a range of 1 to 30 weight %.

In the virus-inactivating agent according to the present invention, in addition to the above-described components, known components that can be blended in a microbe eradicating agent may be contained at suitable amounts in ranges at which the effects of the present invention are not impaired. Examples of such components include antiseptics, antioxidants, surfactants, pigments, fragrances, dyes, etc.

A pH of the virus-inactivating agent according to the present invention is preferably 11 to 14 and more preferably 11 to 13 from the standpoint of the virus-inactivating effect or the microbe eradicating effect against food poisoning microbes.

The virus-inactivating agent according to the present invention is especially significant in the norovirus-inactivating effect and the influenza A virus-inactivating effect and is furthermore excellent in the microbe eradicating effect against general food poisoning microbes. In accordance with concentrations of the above-described blended components, the present invention may be used as an aqueous solution type in which the respective components are practically dissolved in water or as an aqueous dispersion type in which a portion of the components is dissolved in water and a portion of the components is dispersed in water. Although the virus-inactivating agent according to the present invention may be used as it is without diluting with water before use, in a case of using an aqueous dispersion type, it is preferable to dilute it with water before use. In employing any usage, such as using an aqueous solution type or an aqueous dispersion type as it is or diluting either with water before use, etc., the blending proportion of the virus-inactivating components in the virus-inactivating agent is preferably such that an actual usage concentration of the components is 0.01 to 5 weight %.

In a case of using an aqueous solution type, the virus-inactivating agent may be filled in a spray container and used by spraying onto a worker's hands, arms, and other skin portions, knives, pots, and other cooking utensils, plates, containers, and other eating utensils, fresh fish and shellfish, vegetables, meats, and other fresh foods, processed foods, etc. , to which the abovementioned viruses may be attached, in a food factory, restaurant kitchen, or household kitchen, etc. In a case of using an aqueous dispersion type, the virus-inactivating agent may be diluted with water before use and may be used by immersing therein the portable objects among the applicable objects included in the examples above.

### EXAMPLES

Although the present invention shall now be described in further detail below by way of test examples, etc. , the present invention is by no means limited thereby.

### 1. Verification of Inactivating Effect Against Feline Calicivirus

### A. Outline of the Test

An aqueous dispersion (the present invention product) of the formulation indicated below was prepared as a test substance by blending calcium hydroxide, ethanol, and sodium lactate and using water as the remaining portion, and a verification test concerning the inactivating effect of the invention product against feline calicivirus was performed at Japan Food Research Laboratories. That is, norovirus itself was not cultured with stability and thus in the present test, feline calicivirus, which was closely related to norovirus in morphological characteristics and genome structure, was used as an alternative virus. Details of the test are as indicated below.

### B. Test Method

### 1) Test Substance: Aqueous Dispersion (The Present Invention Product)

| | |
|---|---|
| Calcium hydroxide | 12.8 weight % |
| Ethanol | 19.7 weight % |
| Sodium lactate | 9.9 weight % |
| Water | 57.6 weight % |

### 2) Test Virus

Feline calicivirus F-9 ATCC VR-782 (feline calicivirus)

### 3) Cells Used

CRFK cells (Dainippon Pharmaceutical Co., Ltd.)

### 4) Media Used

### (1) Cell Growth Medium

Eagle MEM “Nissui” (1) (Nissui Pharmaceutical Co. Ltd.) with fetal bovine serum added at 10%.

### (2) Cell Maintenance Medium

Eagle MEM “Nissui” (1) (Nissui Pharmaceutical Co. Ltd.) with fetal bovine serum added at 2%.

### 5) Preparation of Virus Suspension

### (1) Culturing of Cells

The cells used were monolayer-cultured in a tissue culture flask using the cell growth medium.

### (2) Inoculation of Virus

After monolayer-culturing, the cell growth medium was removed from the inside of the flask and the test virus was inoculated. The cell maintenance medium was then added and culturing was performed for 1 to 5 days in a carbon dioxide gas incubator (CO₂ concentration: 5%) set at 37° C.

### (3) Preparation of Virus Suspension

After culturing, the cell morphology was observed using an inverted phase contrast microscope to confirm that morphological changes of cells (cytopathic effect) occurred. The culture solution was then centrifuged (3,000 r/min, for 10 minutes) and the resulting supernatant liquid was used as the virus suspension.

### 6) Test Procedure

Using purified water, a 2% suspension of the test substance was prepared as a test liquid. After adding 0.1 ml of the virus suspension to lml of the test liquid and mixing, the mixture was stored under room temperature while shaking at 80 to 90 r/min, and at 6 and 24 hours later, the test liquid was diluted by 100 times using the cell maintenance medium.

As a control test liquid, the purified water was also subject to the test in the same manner. However, with the purified water, measurement was also made at the start time.

### 7) Measurement of Virus Infectivity Titer

The cells used were monolayer-cultured using the cell growth medium in a tissue culture microplate (96-well) and thereafter, the cell growth medium was removed and 0.1 ml of the cell maintenance medium was added to each well. 0.1 ml of the diluted test liquid was then inoculated into each of four wells and culturing was performed for 4 to 7 days in a carbon dioxide gas incubator (CO₂ concentration: 5%) set at 37° C. After culturing, an inverted phase contrast microscope was used to observe whether or not morphological changes of cells (cytopathic effect) occurred, and a median tissue culture infectious dose (TCID₅₀) was calculated by the Reed-Muench method and converted to a virus infectivity titer per 1 ml of test liquid. The results are shown in Table 1.

TABLE 1

| | | log TCID₅₀/ml*¹ | | |
|---|---|---|---|---|
| | | | 6 hours | 24 hours |
| Test virus | Subject | Start time | later | later |
| Feline | Test | 8.5 | <2.5 | <2.5 |
| calicivirus | substance | | | |
| | Control | 8.5 | 8.2 | 7.8 |

TCID₅₀: median tissue culture infectious dose
*¹Logarithmic value of TCID₅₀ per 1 ml of the test liquid
Start time: The TCID₅₀ of the control which was measured immediately after the onset of action was set as that was measured at the start time.
Control: Purified water
Action conditions: Storage while shaking under room temperature
<2.5: Not detected

Table 1 shows that the invention product completely inactivated feline calicivirus of 3.1×10⁸ TCID₅₀/ml within 6 hours.

### 2. Verification of Inactivating Effect Against Norovirus

(1) An aqueous solution (invention product) of the formulation indicated below was prepared as a test substance by blending calcium hydroxide, ethanol, and sodium lactate and using water as the remaining portion, and a verification test concerning the inactivating effect of the invention product against norovirus was performed at the Food Analysis Center of Visionbio Corporation.

### <Test Substance: Aqueous Solution (Invention Product)>

| | |
|---|---|
| Calcium hydroxide | 0.26 weight % |
| Ethanol | 10 weight % |
| Sodium lactate | 5 weight % |
| Water | 84.74 weight % |

A norovirus detection test was performed in accordance with the “Norovirus Detection Method” (latest revision: Food Safety Monitor Issue Report No. 0514004, May 14, 2007) published by the Japanese Ministry of Health, Labor, and Welfare. Fecal norovirus (belonging to NV genogroup II) was used as the norovirus. As the experimental procedure, each of the four samples indicated below was stirred by a vortex mixer, RNA extraction was started after letting stand for 10 minutes, DNase treatment and RT-PCR reaction (reverse transcriptase polymerase chain reaction) were performed, and the PCR product obtained was subject to 2.5% agarose gel electrophoresis. After the end of electrophoresis, gel staining and photography were performed and bands were checked. In the RT-PCR reaction, G2-SKF/G2-SKR (amplification fragment; 344 bp) and G2-SKF/G2AL-SKR (amplification fragment; 344 bp) were used as primers for genogroup (G) II detection. An electrophoretogram of the PCR products is shown in FIG. 1.

### <Samples>

1: Sample without anything added to 500 μl of a norovirus suspension (control group 1)
2: Sample with 400 μl of purified water added to 100 μl of the norovirus suspension (control group 2)
3: Sample with 400 μl of the invention product added to 100 μl of the norovirus suspension (invention product added group 1)
4: Same sample as 3 above (invention product added group 2)

In the present verification test, a band appears at the 344 bp position in the case of a norovirus-positive result. FIG. 1 shows that whereas a band was not seen and the norovirus gene was thus not detected in each of Samples 3 and 4 to which the invention product was added, the band of the norovirus gene was detected in each of Samples 1 and 2 to which the invention product was not added. It was thus confirmed that under the conditions of the present verification test, the invention product has an inactivating effect against norovirus.

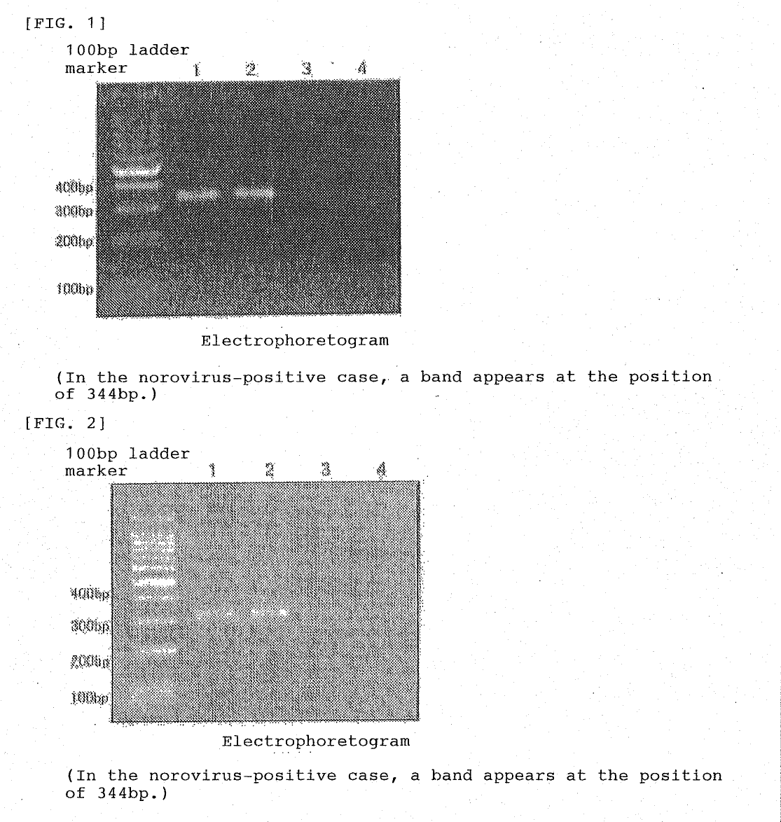

(2) Besides using an aqueous solution (invention product) that was obtained by blending calcium hydroxide with water and not blending in ethanol and sodium lactate in accordance with the formulation shown below as a test substance and stirring each of the four samples indicated below with the vortex mixer and starting RNA extraction after letting stand for 30 minutes, a verification test concerning an inactivation effect against norovirus was performed by the same method as that of (1) above. An electrophoretogram of the PCR products is shown in FIG. 2.

### <Test Substance: Aqueous Solution (Invention Product)>

| | |
|---|---|
| Calcium hydroxide | 0.26 weight % |
| Water | 99.74 weight % |

### <Samples>

1: Sample without anything added to 500 μl of the norovirus suspension (control group 1)
2: Sample with 400 μl of purified water added to 100 μl of the norovirus suspension (control group 2)
3: Sample with 400 μl of the invention product added to 100 μl of the norovirus suspension (invention product added group 1)
4: Same sample as 3 above (invention product added group 2)

FIG. 2 shows that whereas a band was not seen and the norovirus gene was thus not detected in each of Samples 3 and 4 to which the invention product was added, the band of the norovirus gene was detected in each of Samples 1 and 2 to which the invention product was not added. It was thus confirmed that under the conditions of the present verification test, the invention product has an inactivating effect against norovirus.

In comparing the experimental conditions of (1) and (2) above, whereas in (1), the time of treatment against norovirus by the invention product (in which calcium hydroxide, ethanol, and sodium lactate were blended and water was used as the remaining portion) was 10 minutes, in (2), the time of treatment against norovirus by the invention product (in which calcium hydroxide was blended and water was used as the remaining portion) was 30 minutes. And as shown in FIGS. 1 and 2, the band of the norovirus gene was not detected in any of Samples 3 and 4 of (1) and Samples 3 and 4 of (2). It is considered that the time of treatment against norovirus by the invention product is correlated with the level of virus-inactivating effect, and from the results of (1) and (2) above, it is considered that the invention product of (1) is better in terms of virus-inactivating effect than the invention product of (2).

### 3. Verification of Inactivating Effect Against Influenza A Virus

### A. Outline of the Test

The same test substance as that of (1) of “2. Verification of inactivating effect against norovirus” described above was used in performing a verification test concerning the inactivating effect of the invention product against influenza A virus at Japan Food Research Laboratories. Details of the test are as indicated below.

### B. Test Method

### 1) Test Substance: Aqueous Solution (Invention Product)

| | |
|---|---|
| Calcium hydroxide | 0.26 weight % |
| Ethanol | 10 weight % |
| Sodium lactate | 5 weight % |
| Water | 84.74 weight % |

### 2) Test Virus

Influenza A virus (H1N1)

### 3) Cells Used

MDCK (NBL-2) cells ATCC CCL-34 strain (Dainippon Pharmaceutical Co., Ltd.)

### 4) Media Used

### (1) Cell Growth Medium

| | |
|---|---|
| Eagle MEM “Nissui” (1) | 1,000 ml |
| (Nissui Pharmaceutical Co. Ltd.) | |
| 10% NaHCO₃ | 14 ml |
| L-glutamine (30 g/l) | 9.8 ml |
| 100 × vitamin solution for MEM | 30 ml |
| 10% albumin | 20 ml |
| 0.25% trypsin | 20 ml |

### 5) Preparation of Virus Suspension

### (1) Culturing of Cells

The cells used were monolayer cultured in a tissue culture flask using the cell growth medium.

### (2) Inoculation of Virus

After monolayer culturing, the cell growth medium was removed from inside the flask and the test virus was inoculated. The cell maintenance medium was then added and culturing was performed for 1 to 5 days in a carbon dioxide gas incubator (CO₂ concentration: 5%) set at 37° C.

### (3) Preparation of Virus Suspension

After culturing, the cell morphology was observed using an inverted phase contrast microscope to confirm that morphological changes of cells (cytopathic effect) occurred. The culture solution was then centrifuged (3,000 r/min, for 10 minutes) and the resulting supernatant liquid was used as the virus suspension.

### 6) Test Procedure

0.1 ml of the virus suspension was added to and mixed with 1 ml of the test substance to prepare an action solution. The action solution was left to act under room temperature, and 30 seconds, 1 minute, and 5 minutes later, it was diluted by 100 times using the cell maintenance medium. As a control, the purified water was also subject to the test in the same manner and subject to measurement at the start time and at 5 minutes later.

### 7) Measurement of Virus Infectivity Titer

The cells used were monolayer-cultured using the cell growth medium in a tissue culture microplate (96-well) and thereafter, the cell growth medium was removed and 0.1 ml of the cell maintenance medium was added to each well. 0.1 ml of the diluted action solution was then inoculated into each of four wells and culturing was performed for 4 to 7 days in a carbon dioxide gas incubator (CO₂ concentration: 5%) set at 37° C. After culturing, an inverted phase contrast microscope was used to observe whether or not morphological changes of cells (cytopathic effect) occurred, and a median tissue culture infectious dose (TCID₅₀) was calculated by the Reed-Muench method and converted to a virus infectivity titer per 1 ml of action solution. The results are shown in Table 2.

TABLE 2

| | | log TCID₅₀/ml* | | | |
|---|---|---|---|---|---|
| | | | 30 | 1 | 5 |
| Test | | Start | seconds | minute | minutes |
| virus | Subject | time | later | later | later |
| Influenza | Test | 7.3 | 3.5 | <2.5 | <2.5 |
| A virus | substance | | | | |
| | Control | 7.3 | *** | *** | 7.7 |

TCID₅₀: median tissue culture infectious dose
*Logarithmic value of TCID₅₀ per 1 ml of the test liquid
Start time: The TCID₅₀ of the control which was measured immediately after the onset of action was set as that was measured at the start time.
Control: Purified water
Action conditions: Room temperature
<2.5: Not detected
***: Test not performed

Table 2 shows that the invention product completely inactivated influenza A virus of 2.0×10⁷ TCID₅₀/ml within 1 minute.

### 4. Raw Oyster Microbe Eradication Test

Shelled raw oysters were immersed for 12 hours at ordinary temperature in artificial seawater (Sea Water, made by Gex Co., Ltd.) containing 10³ to 10⁴ cfu/ml of a food poisoning microbe (Staphylococcus aureus, Salmonella enteritidis, enterohemorrhagic Escherichia coli O-157, Vibrio parahaemolyticus, or Escherichia coli) (immersion step), the contaminated raw oysters were then taken out after immersion, immersed at ordinary temperature in another artificial seawater containing the microbe eradicating agent indicated below (microbe eradicating step), and the viable bacterial count of the contaminated raw oysters at the point of immersion and 3, 6, 9, and 24 hours later were measured to examine the microbe eradicating effect of the microbe eradicating agent.

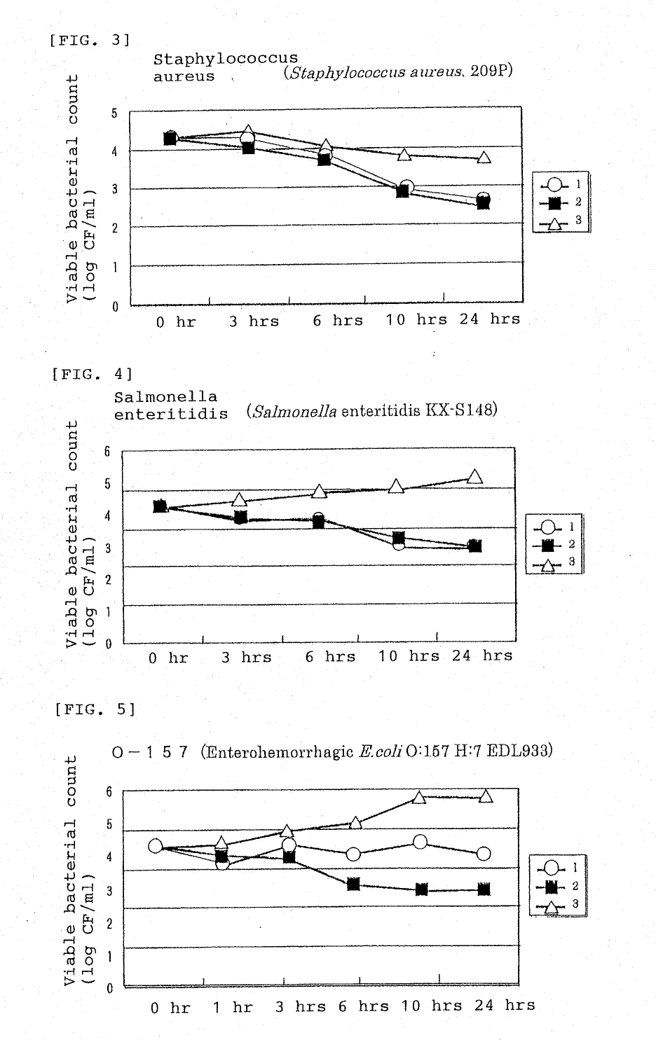

As controls, the same test as described above was performed using a test group of shelled raw oysters immersed in artificial seawater without food poisoning microbe added in the immersion step and using a test group of contaminated raw oysters immersed in artificial seawater without the microbe eradicating agent added in the microbe eradicating step. The results are shown in FIG. 3 to FIG. 8.

### <Test Groups of the Microbe Eradicating Step>

1: 1 ppm of sodium hypochlorite is contained in the artificial seawater.
2: 2 weight % of the test substance (the invention product) used in “1. Verification of an inactivating effect against feline calicivirus” is contained in artificial seawater.
3: Artificial seawater alone (without any microbe eradicating agent added)

FIG. 3 to FIG. 8 show that Test Group 2, in which the invention product was blended, indicates a microbe eradicating effect equal to or greater than that indicated in Test Group 1 in which sodium hypochlorite was blended.

### 5. Sterilization Effects of Compositions Made of Calcinated Calcium and Ethanol

Compositions (invention products) were prepared in which calcinated shell calcium (NC Corporation) and ethanol were blended according to the blending formulations shown in Table 3 and Table 4 and water was used as the remaining portion, and sterilization effects against Escherichia coli (NIHJ) and Staphylococcus aureus (209P) (may hereinafter be referred to as “test bacteria”) were examined.

### (Sterilization Effect Determination Test)

The sterilization effects of the above compositions were determined by Fujimoto's modified method (“Bokin Bobai” (Antibacterial and antifungal agents), Gihodo Shuppan, pp. 683-684) of the Kelsey-Sykes method (The pharmaceutical journal, Nov. 30, 1974 issue, pp. 528-530), which is known as a method for determining the sterilization effects of disinfectants. In outline, the operation procedure is as follows.

(1) 3 ml of the mixed solution or aqueous dispersion prepared as described above was dispensed into a reaction container set at 20° C., 1 ml of the test bacteria adjusted to a concentration of 10⁴ to 10⁵ cfu/ml was added (this point in time is defined as the initial test bacteria addition start time), and 8 minutes later, the reaction solution was sampled and 0.02 ml (1 drop) thereof were added/inoculated into each of five test tubes containing a post-culture medium (Bacto™ Tryptic Soy Broth).

(2) 2 minutes later (after the elapse of 10 minutes from the addition start time of step (1)), 1 ml of the test bacteria was injected into the reaction solution, and 8 minutes later (after the elapse of 18 minutes from the addition start time of step (1)), the reaction solution was sampled and 0.02 ml (1 drop) thereof were added/inoculated into each of five test tubes containing the post-culture medium (Bacto™ Tryptic Soy Broth).

(3) 2 minutes later (after the elapse of 20 minutes from the addition start time of step (1)), 1 ml of the test bacteria was injected into the reaction solution, and 8 minutes later (after the elapse of 28 minutes from the addition start time of step (1)), the reaction solution was sampled and 0.02 ml (1 drop) thereof were added/inoculated into each of five test tubes containing the post-culture medium (Bacto™ Tryptic Soy Broth).

### (Evaluation)

The five test tubes obtained in each of steps (1) to (3) were subject to culturing for 24 hours at 37° C., and if proliferation of the test bacteria was not seen in three or more of the five test tubes of each step, it was judged that there was a sterilization effect.

The evaluation was specifically carried out as follows.

The results are shown in Table 3 and Table 4.
D: There is no sterilization effect.
C: There is a sterilization effect in step (1) but no sterilization effect in step (2).
B: There is a sterilization effect up to step (2) but no sterilization effect in step (3).
A: There is a sterilization effect up to step (3).

TABLE 3
<Bacterial strain used: Escherichia coli>

| | | Ethanol (wt %) | | | | | | | |
|---|---|---|---|---|---|---|---|---|---|
| | | 0 | 5 | 10 | 20 | 30 | 50 | 75 | 100 |
| Calcinated | 0 | D | D | D | D | C | B | B | A |
| calcium (wt %) | 0.05 | C | C | A | A | A | — | — | — |
| | 0.1 | C | B | A | A | A | — | — | — |
| | 0.2 | B | B | A | A | A | — | — | — |
| | 0.4 | A | A | A | A | A | — | — | — |

D: There is no sterilization effect.
C: There is a sterilization effect in step (1) but no sterilization effect in step (2).
B: There is a sterilization effect up to step (2) but no sterilization effect in step (3).
A: There is a sterilization effect up to step (3).
—: Test was not performed (because the effect is obvious).

TABLE 4
<Bacterial strain used: Staphylococcus aureus>

| | | Ethanol (wt %) | | | | | | | |
|---|---|---|---|---|---|---|---|---|---|
| | | 0 | 5 | 10 | 20 | 30 | 50 | 75 | 100 |
| Calcinated | 0 | D | D | D | D | D | D | B | A |
| calcium (wt %) | 0.05 | D | D | D | A | A | — | — | — |
| | 0.1 | D | D | C | A | A | — | — | — |
| | 0.2 | D | C | C | A | A | — | — | — |
| | 0.4 | D | B | B | A | A | — | — | — |

D: There is no sterilization effect.
C: There is a sterilization effect in step (1) but no sterilization effect in step (2).
B: There is a sterilization effect up to step (2) but no sterilization effect in step (3).
A: There is a sterilization effect up to step (3).
—: Test was not performed (because the effect is obvious).

The results of Table 3 show that against Escherichia coli, an adequate sterilization effect (evaluation result: A) was seen when ethanol was blended at no less than 10 weight % in the case where the calcinated calcium was blended at no less than 0.05 weight %. Also, the results of Table 4 show that against Staphylococcus aureus, an adequate sterilization effect (evaluation result: A) was seen when ethanol was blended at no less than 20 weight % in the case where the calcinated calcium was blended at no less than 0.05 weight %.

6. Sterilization Effects of Compositions with Sodium Lactate Blended

Various compositions (invention products) were prepared in which calcinated shell calcium (NC Corporation), ethanol, and sodium lactate (60 weight % aqueous solution, Musashino Chemical Laboratory) were blended according to the blending formulations shown in Table 5 and Table 6 and water was used as the remaining portion, and sterilization effects against Escherichia coli and Staphylococcus aureus were examined. The sterilization effect determination test and evaluation method were carried out in the same manner as in “5. Sterilization effects of compositions made of calcinated calcium and ethanol.” The results are shown in Table 5 and Table 6.

TABLE 5
Sterilization effect against Escherichia coli

| Ethanol (wt %) | | 0 | 5 | 10 |
|---|---|---|---|---|
| Calcinated calcium (wt %) | | 0 | 0.1 | 0.2 |
| Sodium lactate (wt %) | 0 | D | B | A |
| | 1.5 | D | A | — |
| | 3 | D | A | A |
| | 4.5 | D | A | — |
| | 6 | D | — | A |
| | 9 | D | — | A |

D: There is no sterilization effect.
C: There is a sterilization effect in step (1) but no sterilization effect in step (2).
B: There is a sterilization effect up to step (2) but no sterilization effect in step (3).
A: There is a sterilization effect up to step (3).
—: Test was not performed (because the effect is obvious).

TABLE 6
Sterilization effect against Staphylococcus aureus

| Ethanol (wt %) | | 0 | 5 | 10 |
|---|---|---|---|---|
| Calcinated calcium (wt %) | | 0 | 0.1 | 0.2 |
| Sodium lactate (wt %) | 0 | D | D | C |
| | 1.5 | D | D | — |
| | 3 | D | D | C |
| | 4.5 | D | C | — |
| | 6 | D | — | A |
| | 9 | D | — | A |

D: There is no sterilization effect.
C: There is a sterilization effect in step (1) but no sterilization effect in step (2).
B: There is a sterilization effect up to step (2) sterilization effect in step (3).
A: There is a sterilization effect up to step (3).
—: Test was not performed (because the effect is obvious).

The results of Table 5 show that although with sodium lactate alone, no sterilization effect whatsoever was seen against Escherichia coli even when sodium lactate was blended at 9 weight % (see the left column of Table 5), when no less than 1.5 weight % of sodium lactate was blended in the case where 5 weight % of ethanol and 0.1 weight % of calcinated calcium were blended, the sterilization effect evaluation result changed from B to A (see central column of Table 5) . An effect of enhancing the sterilizing activity against Escherichia coli was thus seen when sodium lactate was blended with the composition containing ethanol and calcinated calcium.

This trend was seen significantly in the sterilization effect against Staphylococcus aureus. That is, when no less than 4.5 weight % of sodium lactate was blended in the case where 5 weight % of ethanol and 0.1 weight % of calcinated calcium were blended, the sterilization effect evaluation result changed from D to C (see central column of Table 6) , and when no less than 6 weight % of sodium lactate was blended in the case where 10 weight % of ethanol and 0.2 weight % of calcinated calcium were blended, the sterilization effect evaluation result changed from C to A (see right column of Table 6).

It is presumed that the sterilizing activity enhancement effect of sodium lactate is due to improvement of solubility of calcium hydroxide in the composition. That is, it is generally considered that the calcinated calcium reacts with the water in the composition and is present as calcium hydroxide and that the calcium hydroxide exhibits the sterilization effect. The solubility of calcium hydroxide in water is low and although it is thus considered as not exhibiting its inherent sterilization effect, the above results seem to suggest that the solubility of the produced calcium hydroxide was improved by the addition of sodium lactate.

The virus-inactivating agent according to the present invention is widely usable as a virus-inactivating agent applicable not only to foods per se but also to cooking utensils, cooking equipment, and other food manufacturing equipment.

[FIG. 1]

100 bp ladder marker

Electrophoretogram

(In the norovirus-positive case, a band appears at the position of 344 bp.)

[FIG. 2]

100 bp ladder marker

Electrophoretogram

(In the norovirus-positive case, a band appears at the position of 344 bp.)

[FIG. 3]

Staphylococcus aureus

Viable bacterial count (log CF/ml)

0 hour

3 hours

6 hours

10 hours

24 hours

[FIG. 4]

Salmonella enteritidis

Viable bacterial count (log CF/ral)

0 hour

3 hours

6 hours

10 hours

24 hours

[FIG. 5]

Enterohemorrhagic Escherichia coli 0-157

Viable bacterial count (log CF/ml)

0 hour

1 hour

3 hours

6 hours

10 hours

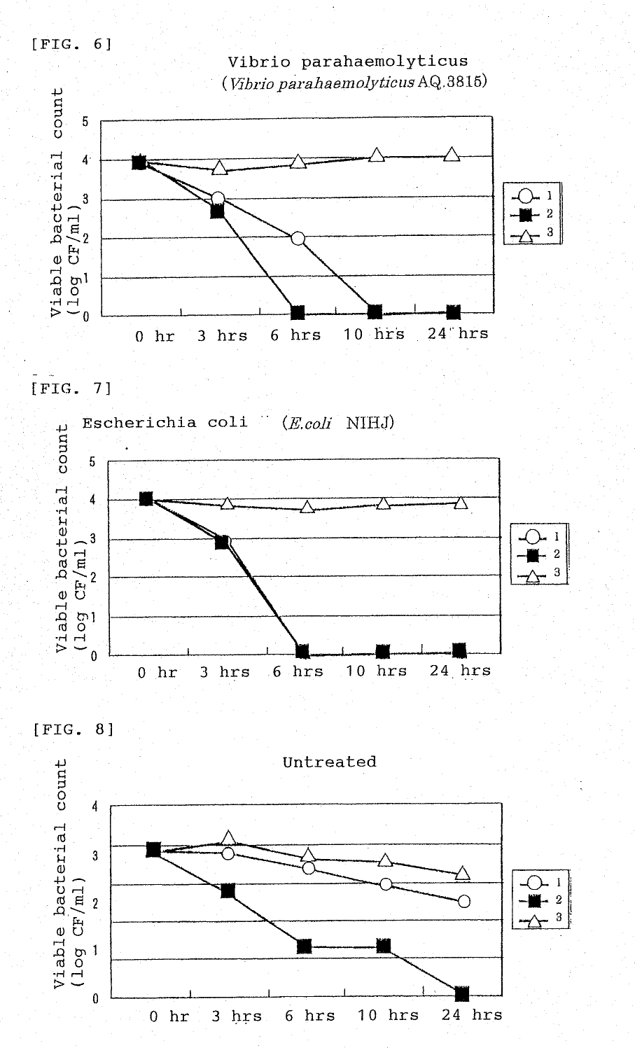

24 hours

[FIG. 6]

Vibrio parahaemolyticus

Viable bacterial count (log CF/ml)

0 hour

3 hours

6 hours

10 hours

24 hours [FIG. 7]

Escherichia coli

Viable bacterial count (log CF/ml)

0 hour

1 hours

3 hours

6 hours

10 hours

24 hours

[FIG. 8]

Untreated

Viable bacterial count (log CF/ml)

0 hour

1 hour

3 hours

6 hours

10 hours

24 hours

1. A norovirus-inactivating agent that is an aqueous solution or aqueous dispersion prepared by blending in calcium hydroxide.

2. An influenza A virus-inactivating agent that is an aqueous solution or aqueous dispersion prepared by blending in calcium hydroxide.

3. The virus-inactivating agent according to claim 1 prepared by blending in calcium oxide and/or calcinated calcium in place of calcium hydroxide or in addition to calcium hydroxide.

4. The virus-inactivating agent according to claim 1 prepared by further blending in ethanol and sodium lactate.

5. The virus-inactivating agent according to claim 4, wherein a total blending proportion of the blended components among calcium hydroxide, calcium oxide, and calcinated calcium is 0.01 to 30 weight %, a blending proportion of ethanol is 5 to 30 weight %, and a blending proportion of sodium lactate is 1 to 30 weight %.

6. The virus-inactivating agent according to claim 1, wherein an average particle diameter of calcium hydroxide, calcium oxide, and calcinated calcium is 0.1 to 10 μm.

7. A food obtained by virus inactivation treatment by the virus-inactivating agent according to claim 1.

8. The food according to claim 7 that is a fresh fish or shellfish.

9. The virus-inactivating agent according to claim 2 prepared by blending in calcium oxide and/or calcinated calcium in place of calcium hydroxide or in addition to calcium hydroxide.

10. The virus-inactivating agent according to claim 2 prepared by further blending in ethanol and sodium lactate.

11. The virus-inactivating agent according to claim 3 prepared by further blending in ethanol and sodium lactate.

12. The virus-inactivating agent according to claim 2, wherein an average particle diameter of calcium hydroxide, calcium oxide, and calcinated calcium is 0.1 to 10 μm.

13. The virus-inactivating agent according to claim 3, wherein an average particle diameter of calcium hydroxide, calcium oxide, and calcinated calcium is 0.1 to 10 μm.

14. The virus-inactivating agent according to claim 4, wherein an average particle diameter of calcium hydroxide, calcium oxide, and calcinated calcium is 0.1 to 10 μm.

15. The virus-inactivating agent according to claim 5, wherein an average particle diameter of calcium hydroxide, calcium oxide, and calcinated calcium is 0.1 to 10 μm.

16. A food obtained by virus inactivation treatment by the virus-inactivating agent according to claim 2.

17. A food obtained by virus inactivation treatment by the virus-inactivating agent according to claim 3.

18. A food obtained by virus inactivation treatment by the virus-inactivating agent according to claim 4.

19. A food obtained by virus inactivation treatment by the virus-inactivating agent according to claim 5.

20. A food obtained by virus inactivation treatment by the virus-inactivating agent according to claim 6.
